(19) United States
(12) Patent Application Publication
(10) Pub. No.: US 2024/0376211 A1
(43) Pub. Date: Nov. 14, 2024

(54) A PROCESS TO PRODUCE A PHARMACEUTICAL COMPOSITION

(71) Applicant: DR. REDDY’S LABORATORIES LIMITED, Hyderabad (IN)

(72) Inventors: Akhila PUDIPEDDI, Visakhapatnam (IN); L C Pavani PARUPUDI, East Godavari District (IN); Saswat SWAYAMSIDDH, Bhubaneswar (IN); Sneha KANNAN, Hyderabad (IN); Suman BANDYOPADHYAY, Secunderabad (IN)

(21) Appl. No.: 18/685,082

(22) PCT Filed: Aug. 19, 2022
(86) PCT No.: PCT/IN2022/050750

§ 371 Date: Feb. 20, 2024

(30) Foreign Application Priority Data
Aug. 20, 2021 (IN) 202141037886

(51) Int. Cl. C07K 16/28

(52) U.S. Cl. CPC C07K 16/2839; C07K 2317/24

(57) ABSTRACT

The present invention relates to a cell culture process for producing a monoclonal antibody composition, the process comprising culturing the mammalian cells at a pH range of about 6.6 to about 7.5, performing a temperature shift from a first culture temperature to a second culture temperature, supplementation of galactose in the cell culture, thereby obtaining an antibody composition comprising galactosylated glycoform and/or G0F glycoform. Further, the cell culture process disclosed in the present invention obtains an antibody composition comprising galactosylated glycoform wherein the percentage of galactosylated glycoform decreases with the decrease in the difference between the first temperature and the second temperature.

### FIELD OF INVENTION

The Biological material used in the invention was not obtained from India.

The present invention relates to a cell culture process for culturing mammalian cells producing a pharmaceutical composition. The invention provides a mammalian cell culture process comprising culturing the mammalian cells at a pH range of 6.6 to 7.5, performing a temperature shift and supplementation of galactose, to obtained a target/predetermined pharmaceutical composition. In particular, the invention provides a cell culture process to obtain an antibody composition comprising galactosylated glycoform and/or G0F glycoform.

### BACKGROUND OF THE INVENTION

Monoclonal antibodies (mAbs) are the fastest growing pharmaceutical products used for diseases like cancer, autoimmune, cardiovascular and inflammatory disorders. They are produced in mammalian cells like Chinese hamster ovary (CHO) cells since they have post-translational modifications (PTMs) which are integral to the effector function of the therapeutic antibody molecules. PTMs refer to enzymatic modification of proteins following its translation which generally result in mature protein/antibody. The PTMs can include addition of chemical moeities to target proteins which range from addition of simple smaller groups as in phosphorylation, methylation, acetylation, hydroxylation, to addition of complex biomolecules as in glycosylation, prenylation etc. or addition of polypeptide as in ubiquitination. The PTMs can result in modification of amino acid residues in protein and also result in proteolytic degradation of proteins. As a result of these modifications, the structure, stability and function of the proteins are altered.

Amongst various PTMs in therapeutic antibodies, glycosylation has significant effect on properties relevant to the therapeutic applications of antibodies such as biological activity, efficacy, stability, immunogenicity, clearance rate, antibody-dependent cellular cytoxicity (ADCC), and complement-dependent cytoxicity (CDC).

The two major types of glycosylation in mammalian expression systems are N-linked glycosylation, in which glycans are attached to the asparagine of the recognition sequence Asn-X-Thr/Ser, where “X” is any amino acid except proline, and O-linked glycosylation in which glycans are attached to serine or threonine. The N-linked glycosylation results in heterogeneity in antibody glycoforms which affect the efficacy and safety of the antibody. Hence various studies have been done to understand and characterize the glycoform distributions in antibodies during their productions (Radhakrishnan, D., A. S. Robinson, and B. A. Ogunnaike, Controlling the Glycosylation Profile in mAbs Using Time-Dependent Media Supplementation. Antibodies, 2017. 7(1): p. 1)

Vedolizumab, a recombinant humanized IgG1 monoclonal antibody binding to human α4β7 integrin, is approved for the treatment of moderate to severely active ulcerative colitis and Crohn's disease in adults who have failed at least one conventional therapy. It consists of an asparagine-linked glycosylation site on each of the heavy chain. (Wyant et. al. An Overview of the Mechanism of Action of the Monoclonal Antibody Vedolizumab. Journal of Crohn's and Colitis, 2016, 1437-1444).

‘Biosimilars’, sometimes called ‘similar biological medicinal product’ or ‘follow-on biologic’ or ‘subsequent entry biologic’, include therapeutic mAbs which are similar to already licensed therapeutic product (the ‘reference product’). Regulatory agencies mandate that biosimilars must demonstrate high similarity to the reference product, in terms of quality characteristics, biological activity, safety and efficacy, in order to have marketing approval (Sullivan, P. M. and L. M. DiGrazia, Analytic characterization of biosimilars. Am J Health Syst Pharm, 2017. 74(8): p. 568-579; Guttman et. al. Assessing Glycosimilarity of Biotherapeutics. 2018 https://sciex.com/content/dam/SCIEX/pdf/tech-notes/all/Glycosimilarity.pdf).

Given the importance of glycosylation in the regulatory approval of reference products and biosimilars alike, much efforts have been undertaken to understand the cell culture process parameters which may have a bearing on the glycosylation pattern of the therapeutic products. Previous studies have demonstrated that glycosylation of mAbs can be influenced by various factors such as pH, temperature, dissolved oxygen, ammonia, and media supplements such as nucleotide sugar precursors and manganese chloride. These studies focused on individual factors, establishing empirical relationships between the individual factor in question and the specific set of glycoform species it affects (Radhakrishnan, D., A. S. Robinson, and B. A. Ogunnaike, Controlling the Glycosylation Profile in mAbs Using Time-Dependent Media Supplementation. Antibodies, 2017. 7(1): p. 1).

Therefore, the present invention relates to a cell culture process for producing an antibody composition, the process comprising culturing mammalian cells at a pH range of 6.6 to 7.5, performing a temperature shift from a first culture temperature to a second culture temperature, supplementation of galactose in the cell culture, thereby obtaining an antibody composition comprising galactosylated glycoform and G0F glycoform, wherein the percentage of galactosylated glycoform decreases with the decrease in the difference between the first temperature and the second temperature.

### SUMMARY OF THE INVENTION

The present invention relates to a cell culture process for producing a pharmaceutical monoclonal antibody composition, the process comprising culturing mammalian cells expressing the monoclonal antibody at a pH range of about 6.6 to about 7.5, performing a temperature shift from a first culture temperature to a second culture temperature, supplementation of galactose in the cell culture, thereby obtaining an antibody composition comprising galactosylated glycoform and/or G0F glycoform. Further, the present invention discloses that the obtained antibody composition comprises galactosylated glycoform wherein the percentage of galactosylated glycoform decreases with the decrease in the difference between the first temperature and the second temperature.

### DETAILED DESCRIPTION OF THE INVENTION

### Definitions

The term “about” refers to a range of values that are similar to the stated reference value to a range of values that fall within 25, 20, 19, 18, 17, 16, 15, 14, 13, 12, 11, 10, 9, 8, 7, 6, 5, 4, 3, 2, 1 percent or less of the stated reference value.

The term “antibody” or “monoclonal antibody” refers to an intact antibody or an antigen binding fragment thereof.

The term “antibody composition” refers to a population of antibody molecules or fragments thereof that is produced by mammalian cell culture. The population of antibody molecules may have one or several post translational modifications (PTM), imparting the antibody molecules a different molecular weight, charge, solubility or combinations thereof.

The term “biosimilar” refers to a recombinant pharmaceutical protein, commonly with identical amino acid sequence to a reference product that contains, similar, very similar to or same post-translational modifications as the reference product yielding no clinically meaningful difference in terms of safety, purity and potency.

The term “cell culture process” as used herein refers to a process of culturing a population of cells that are capable of producing recombinant protein of interest or antibody.

The term “G0F glycoform” or “G0F” refers to antibodies with fucose linked to the non-reducing end of N-acetlyglucosamine, and does not contain any terminal galactose residues.

The term “galactosylated glycoform” or “GAL” refer to antibodies containing terminal galactose residues such as G1A, G1B, G1AF, G1BF, G2, G2F and G2SF.

The term “glycan” refers to monosaccharide or polysaccharide moiety attached to another molecule.

The term “glycoform” or “glycovariant” used interchangeably herein refers to different molecular variants of an antibody resulting due to variable glycan structure attached and/or glycan attachment site occupancy on the antibody.

The term “high mannose glycoform” or “HM” refers to antibodies containing unsubstituted terminal mannose sugars. High mannose glycoforms contain more than 4 mannose residues attached to the GlcNAc2 core.

The term “shift in the pH range” refers to the change in range of the pH maintained during the cell culture process.

The term “reference product” refers to a currently or previously marketed recombinant protein, also described as the “originator product” or “branded product” serving as a comparator in the studies.

The term “supplementing” or “supplementation” as used herein refers to any addition made to cell culture medium/feed to achieve the goals described in this disclosure.

The term “target/predetermined levels” refers to the glycosylation levels of the ‘reference product’.

The term “temperature shift” refers to the change in temperature during the cell culture process.

The term “total afucosylated glycans” or “TAF” described here, consists of glycan moieties wherein fucose is not linked to the non-reducing end of N-acetlyglucosamine. Without limitation examples of afucosylated glycans include G0, G1A, G1B, G2, M3-M9NAG, M3-M9.

### DETAILED DESCRIPTION OF THE EMBODIMENTS

The present invention provides a cell culture process for culturing mammalian cells expressing an antibody, the process comprising culturing the cells at a pH range of 6.6 to 7.5 by lowering of temperature of the cell culture from a first temperature to a second temperature, and supplementation of galactose to obtain antibody composition comprising galactosylated variants.

Any mammalian cell or cell type which is suitable for expression of recombinant proteins in a cell culture medium may be used for the present invention. Non-limiting examples of mammalian cells that may be used with the present invention include Chinese hamster ovary (CHO) cells, baby hamster kidney (BHK21) cells and murine myeloma cells (NS0 and Sp2/0) human retinoblasts (PER.C6 cell line), human embryonic kidney cell line (HEK-293 cell line) (Dumont, J., et al., Human cell lines for biopharmaceutical manufacturing: history, status, and future perspectives. Crit Rev Biotechnol, 2016. 36(6): p. 1110-1122). In a preferred embodiment, CHO cell lines expressing recombinant proteins may be used in accordance with the present invention.

Certain glycosylation profile of the pharmaceutical monoclonal antibody (mAb) are desirable based on its mechanism of action as the glycosylation profile effects the stability, safety and efficacy of the antibody. In an embodiment, the cell culture process of the present invention may be used to produce an antibody composition comprising galactosylated glycoform and/or G0F glycoform at a target/predetermined range.

Cell culture medium is understood by those skilled in the art to refer to a nutrient solution in which cells, such as animal or mammalian cells, are grown. A cell culture medium generally includes one or more of the following components: an energy source (e.g., a carbohydrate such as glucose); amino acids; vitamins; lipids or free fatty acids; and trace elements, e.g., inorganic compounds or naturally occurring elements in the micromolar range. Cell culture medium can also contain additional components, such as hormones and other growth factors (e.g., insulin, transferrin, epidermal growth factor, serum, and the like); salts (e.g., calcium, magnesium and phosphate); sugars (e.g. mannose, galactose, fucose); amino acids (glutamine); buffers (e.g., HEPES); nucleosides and bases (e.g., adenosine, thymidine, hypoxanthine); antibiotics (e.g., gentamycin); and cell protective agents (e.g., a Pluronic polyol (Pluronic F68). Commercially available media can be utilized in accordance with the present invention, for example, Dulbecco's Modified Eagles Medium (DMEM, Sigma-Aldrich); RPMI-1640 Medium (Sigma-Aldrich); EX-CELL® Advanced CHO Fed-batch Medium (Sigma-Aldrich); Cell Boost™ 7a and 7b (GE Healthcare Bio-Sciences AB). One skilled in the art would appreciate that some cell culture media are suited to support cells through their initial growth phase (basal medium) while some sustain cells through the later growth phase and production phase of cell culture (feed medium), and would be able to choose appropriate culture medium.

The methods described in the present invention are in recognition of the fact that various parameters of the cell culture process may be used to obtain antibody composition of desired glycosylation profile. In an embodiment, the cell culture process envisages culturing the cells at a pH range of about 6.6 to about 7.5, performing a temperature shift and supplementation of galactose.

A person of ordinary skill in the art would be able to characterize and analyse the various antibody variants present in the antibody composition produced by the cell culture process described herein using the state of the art techniques.

In an embodiment, the present invention discloses a cell culture process for producing an antibody composition, wherein the said process comprises
a) providing mammalian cells expressing the said antibody
b) culturing the cells at a pH range of about 6.6 to about 7.5
c) performing a temperature shift during the cell culture from a first culture temperature to a second culture temperature, wherein the second culture temperature is lower than the first culture temperature
d) supplementing culture media with galactose
e) recovering the said recombinant antibody composition
wherein the antibody composition produced comprises of galactosylated glycoform and/or G0F glycoform, and wherein the percentage of galactosylated glycoform in the antibody composition decreases with the decrease in the difference between the first culture temperature and the second culture temperature.

In an embodiment, the present invention discloses a cell culture process for producing an antibody composition, wherein the said process comprises
a) providing mammalian cells expressing the said antibody
b) culturing cells at a pH range of about 6.6 to about 7.5
c) performing a temperature shift during the cell culture from a first culture temperature to a second culture temperature, wherein the second culture temperature is lower than the first culture temperature
d) supplementing culture media with galactose
e) recovering the said recombinant antibody composition
wherein the antibody composition produced comprises of galactosylated glycoform and/or G0F glycoform, and wherein the percentage of galactosylated glycoform in the antibody composition decreases with the decrease in the difference between the first temperature and the second temperature.

In another embodiment, the present invention discloses a cell culture process for producing an antibody composition, wherein the said process comprises
a) providing/culturing the mammalian cell expressing the said antibody
b) culturing the cells at a pH range of about 6.6 to about 7.5
c) performing a temperature shift during the cell culture from a first culture temperature to a second culture temperature, wherein the second culture temperature is lower than the first culture temperature
d) supplementing culture media galactose
e) recovering the said recombinant antibody composition
wherein the obtained antibody composition is comprised of about 41% to about 47% galactosylated glycoform and/or about 47% to about 52% G0F glycoform.

In an embodiment, the present invention discloses a cell culture process for producing a monoclonal antibody composition having target/predetermined levels of the reference product of the antibody variants, wherein the cell culture process comprises
a) providing/culturing the mammalian cell expressing the said antibody
b) culturing the cells at a pH range of about 6.6 to about 7.5
c) performing a temperature shift during the cell culture from a first culture temperature to a second culture temperature, wherein the second culture temperature is lower than the first culture temperature
d) supplementing the cell culture with galactose
e) recovering the said recombinant antibody composition
wherein the antibody composition produced comprises of galactosylated glycoform, and wherein the percentage of galactosylated glycoform in the antibody composition decreases with the decrease in the difference between the first temperature and the second temperature.

In an embodiment, the present invention discloses a cell culture process for producing a monoclonal antibody composition having target/predetermined levels of the reference product of the antibody variants, wherein the cell culture process comprises
a) providing/culturing the mammalian cell expressing the said antibody
b) culturing the cells at a pH range of about 6.6 to about 7.5
c) performing a temperature shift during the cell culture from a first culture temperature to a second culture temperature, wherein the second culture temperature is lower than the first culture temperature
d) supplementing the cell culture with galactose
e) recovering the said recombinant antibody composition
wherein the antibody variants in the said composition comprised of about 41% to about 47% galactosylated glycoform and/or about 47% to about 52% G0F glycoform.

In any of the embodiments mentioned above, the antibody composition further comprises of afucosylated variants, high mannose variants and total afucosylated variants.

In any of the embodiments mentioned above, the cells are cultured at a pH range of 6.7 to 7.2 till day 4, performing a shift in pH range whereby the cells are cultured thereafter at a pH range of 6.7 to 7.4

In any of the embodiments mentioned above, the temperature shift is performed after the aforementioned shift in pH range.

In any of the embodiments mentioned above, the temperature shift is performed on day 5 of the cell culture, day 6 of the cell culture, or day 7 of the cell culture. In yet another embodiment, the temperature shift is performed on day 6 of the cell culture.

In any of the embodiments mentioned above, the difference between the first culture temperature and the second culture temperature of the cell culture ranges from about 6.5 to 2.5. In another embodiment, the culture temperature before the temperature shift is about 37° C. and the culture temperature after the temperature shift is about 30° C.-35° C. In yet another embodiment, the culture temperature before the temperature shift is about 37° C. and the culture temperature after the temperature shift is selected from about 30° C., about 31° C., about 32° C., about 33° C., about 34° C., about 35° C.

In any of the embodiments mentioned above, the cumulative galactose supplementation in the present invention is 6 g/L, wherein galactose is supplemented as 3 g/L each on day 7 and 10.

In yet another embodiment, the antibody produced using the present invention is an anti α4β7 integrin antibody. In a preferred embodiment, the antibody produced using the present invention is vedolizumab.

Those skilled in the art will recognize that several embodiments are possible within the scope and spirit of this invention. The invention will now be described in greater detail by reference to the following non-limiting examples. The following examples further illustrate the invention but, of course, should not be construed as in any way limiting its scope.

### EXAMPLES

### Example I

An anti-α4β7 integrin antibody having a heavy chain and light chain as described in WO2007061679A1 was cloned and expressed in a recombinant CHO (rCHO) cell line using techniques described in detail in “Molecular Cloning: A Laboratory Manual (Fourth Edition)”. rCHO cells expressing the antibody were seeded at a density of about 0.5 million cells/mL in basal cell culture medium and cultured at an initial temperature of 36.5° C. The pH range of the cell culture is maintained between pH 6.6 to 7.5. The cell culture medium was supplemented with glucose. On day 6, a temperature shift was performed, thereby reducing the culture temperature to 30° C. The cell culture was supplemented with 3 g/L galactose each on day 7 and day 10. Feed medium was added on day 3, 5, 7, 9 and 11. The culture was harvested on day 13. The antibody composition and titer obtained is depicted in Table 1.

### Example II

The cell culture process described in Example 1 was carried with following modifications. The temperature shift applied on day 6 reduced the culture temperature to 32° C. The antibody composition and titer obtained is depicted in Table 1.

### Example III

The cell culture process described in Example 1 was carried with following modifications. The temperature shift applied on day 6 reduced the culture temperature to 34° C. The antibody composition and titer obtained is depicted in Table 1.

### Example IV

The cell culture process described in Example 1 was carried with following modifications. The cells were cultured at a pH 6.7 to 7.2 till day 4 and at pH 6.7 to 7.4 thereafter. The temperature shift applied on day 6 reduced the culture temperature to 32° C. The antibody composition and titer obtained is depicted in Table 1.

TABLE 1
The composition and titer of the anti-α4β7 integrin antibody.

| Examples | GAL (%) | G0F (%) | Titer (g/L) |
|---|---|---|---|
| I | 46.9 | 47.50 | 3.6 |
| II | 43.89 | 49.75 | 4.1 |
| III | 41.58 | 51.57 | 4.2 |
| IV | 43.5 | 48.9 | 3.7 |

1. A cell culture process for producing an antibody composition comprising galactosylated and GOF glycoforms, wherein the said process comprises:
a) providing mammalian cells expressing the said antibody;
b) culturing the mammalian cells in a culture media at a pH range of about 6.6 to about 7.5;
c) performing a temperature shift during cell culture from a first culture temperature to a second culture temperature, wherein the second culture temperature is about 2.5° C. to 6.5° C. lower than the first culture temperature;
d) supplementing the culture media with galactose; and
e) recovering the said recombinant antibody composition,
wherein the percentage of galactosylated glycoform in the antibody composition decreases with the decrease in the difference between the first culture temperature and the second culture temperature.

2. A cell culture process of claim 1, wherein the antibody composition obtained comprises of about 41% to about 47% galactosylated glycoform and about 47% to about 52% GOF glycoform.

3. A cell culture process for producing a monoclonal antibody composition having target/predetermined levels of the reference product of the antibody variants, wherein the cell culture process comprises:
a) providing mammalian cells expressing the said antibody;
b) culturing the mammalian cells in a culture media at a pH range of about 6.6 to about 7.5;
c) performing a temperature shift during cell culture from a first culture temperature to a second culture temperature, wherein the second culture temperature is lower than the first culture temperature;
d) supplementing the culture media with galactose; and
e) recovering the said recombinant antibody composition,
wherein the antibody variants in the said composition comprises of about 41% to about 47% galactosylated glycoform and about 47% to about 52% GOF glycoform.

4. The cell culture process of claim 3, wherein the difference between the first culture temperature and the second culture temperature of the cell culture ranges from about 6.5° C. to 2.5° C.

5. The cell culture process of claim 1, wherein the first culture temperature before the temperature shift is about 37° C. and the second culture temperature after the temperature shift is selected from a range of about 30° C. to about 35° C.

6. The cell culture process of claim 1, wherein the first culture temperature before the temperature shift is about 36.5° C. and the second culture temperature after the temperature shift is selected from about 30° C., about 32° C., about 34° C.

7. A cell culture process of claim 1, wherein the galactose supplemented is about 6 g/L.

8. A cell culture process of claim 1, wherein the antibody composition obtained is an anti α4β7 integrin antibody composition, vedolizumab.

9. A cell culture process of claim 3, wherein the antibody composition obtained is an anti α4β7 integrin antibody, vedolizumab.

10. The cell culture process of claim 2, wherein the first culture temperature before the temperature shift is about 37° C. and the second culture temperature after the temperature shift is selected from a range of about 30° C. to about 35° C.

11. The cell culture process of claim 3, wherein the first culture temperature before the temperature shift is about 37° C. and the second culture temperature after the temperature shift is selected from a range of about 30° C. to about 35° C.

12. The cell culture process of claim 2, wherein the first culture temperature before the temperature shift is about 36.5° C. and the second culture temperature after the temperature shift is selected from about 30° C., about 32° C., about 34° C.

13. The cell culture process of claim 3, wherein the first culture temperature before the temperature shift is about 36.5° C. and the second culture temperature after the temperature shift is selected from about 30° C., about 32° C., about 34° C.

14. The cell culture process of claim 2, wherein the galactose supplemented is about 6 g/L.

15. The cell culture process of claim 3, wherein the galactose supplemented is about 6 g/L.

16. A cell culture process of claim 4, wherein the galactose supplemented is about 6 g/L.

17. The cell culture process of claim 5, wherein the galactose supplemented is about 6 g/L.

18. The cell culture process of claim 6, wherein the galactose supplemented is about 6 g/L.

19. The cell culture process of claim 5, wherein the antibody composition obtained is an anti α4β7 integrin antibody composition, vedolizumab.

20. The cell culture process of claim 6, wherein the antibody composition obtained is an anti α4β7 integrin antibody composition, vedolizumab.
